(19) United States
(12) Patent Application Publication
(10) Pub. No.: US 2024/0122824 A1
(43) Pub. Date: Apr. 18, 2024

(54) ORAL COMPOSITIONS

(71) Applicant: Michael ARNOLD, Beverly Hills, CA (US)

(72) Inventor: Michael ARNOLD, Beverly Hills, CA (US)

(21) Appl. No.: 18/486,879

(22) Filed: Oct. 13, 2023

Related U.S. Application Data
(60) Provisional application No. 63/379,403, filed on Oct. 13, 2022.

(51) Int. Cl. A61K 8/19; A61Q 11/00

(52) U.S. Cl. CPC A61K 8/19; A61Q 11/00; A61K 2800/92

(57) ABSTRACT

The present disclosure provides a chewable composition for teeth-whitening, comprising a first chewable composition and a second chewable composition, that when combined in the mouth and actuated by mastication provide cosmetic enhancement and chemotherapeutic activity. In some aspects, the first chewable composition comprises carbamide peroxide having water content of about 2% or less, an anhydrous chewing gum base having water content of about 2% or less, and tableting excipients having a water content of 2% or less; and the second chewable composition comprises a gum base, a source of bicarbonate ion, and one or more gum additives. The disclosure also provides a kit comprising the first and second separate chewable compositions, and methods for whitening teeth and freshening the breath, and also for reducing the levels of the oral pathogen sources of periodontal diseases and halitosis using the compositions and kits described herein.

### CROSS-REFERENCE TO RELATED APPLICATION

This application claims the benefit of U.S. Provisional Application No. 63/379,403, which was filed on Oct. 13, 2022, and is expressly incorporated by reference herein in its entirety.

### TECHNICAL FIELD

The present disclosure relates to novel oral care compositions and methods for their use for cosmetic as well as chemotherapeutic purposes. More particularly, the disclosure relates to chewing gums for teeth-whitening, oral cleaning and oral microbiological health.

### BACKGROUND

Teeth whiteners, also known as teeth bleaching agents, are in widespread use as a cosmetic means to enhance appearance and to contribute to better oral health and hygiene in general. Among teeth whitening formulations, those containing oxidizing agents as active ingredients are preferred because of their fast action and superior efficacy. The most frequently employed oxidizer in teeth whitening compositions is a peroxide, either in free form as hydrogen peroxide, or as a non-covalent adduct with urea. Such an adduct is known as carbamide peroxide (or urea hydrogen peroxide). It has been well established that the higher the concentration of peroxide, the faster the whitening effect is achieved. However, it is important to note that the higher the concentration of peroxide, the greater the risk of gum and teeth root sensitivity, which has proven a significant problem for at least 10-15% of the population. Also, the higher the concentration of peroxide in the teeth-whitener, the higher the probability that the teeth-whitener will not be stable or have suitable shelf life for commercialization.

In general, stability is in direct conflict with the purpose of the peroxide-based teeth whitening compositions, which is to achieve the best possible whitening in a reasonable length of time of contact with the tooth surface. To accomplish this, the peroxide moiety must be short-lived (on the order of seconds) in the mouth, decomposing into an “active oxygen moiety” which is the ultimate effective teeth-whitening agent. The requirement for quick decomposition in the mouth to generate the active oxygen moiety is in conflict with the need for long shelf life necessary for commercialization. The greater the stabilization, the slower the reactivity of the peroxide moiety, and the less effective the teeth-whitening action.

To improve on the rate of teeth-bleaching action of shelf-stable peroxide-based teeth-whiteners in the mouth, “activators” have been developed. For example, activation by use of UV light has been demonstrated by U.S. Pat. Nos. 5,713,738 and 5,645,428 to Yarborough. For hydrogen peroxide, which is known to decompose in alkaline media, basic compounds and alkaline systems have also been disclosed as activators for hydrogen peroxide teeth-whiteners. For example, Allred, U.S. Pat. No. 6,746,664, describes a variety of alkaline activation systems for whitening teeth. These alkaline agents are provided as a separate component that, when admixed with the hydrogen peroxide composition, reduces the time for hydrogen peroxide to react. However, it is known professionally that many of these teeth-whitening gels and gels with alkaline activators cause extreme sensitivity to the hard and soft tissue. Hydrogen peroxide is known to decompose in the presence of alkaline media. It is believed that the more rapid bleaching of the teeth is a direct consequence of the action of these “activator” components. These alkaline agents typically include pH modifiers, including metal carbonates, metal bicarbonates, phosphates, amines and others. It is important to note, that even though the pH is controlled at a safe level by the addition of an alkaline-based “activator,” effectiveness of these activators in enhancing teeth-whitening reactivity of a hydrogen peroxide teeth-whitener system is questionable since they have not been shown to be better than the corresponding non-activated hydrogen peroxide teeth-whitening compositions (typically gels or pastes). Further, it should be noted that no significant difference in activation or teeth-whitening benefits has been reported for any metal carbonates or metal bicarbonates. Typically, in prior compositions, a metal carbonate salt such as calcium carbonate, is the preferred activating and pH buffering agent.

Traditional teeth-whitening compositions and devices can be classified in three groups based on the method of delivering the material to tooth surfaces. The first is whitening toothpastes in which bleaching agents are typically incorporated as an additional component in these toothpaste formulations. These have not proven to be effective. The second group contains formulations intended specifically for whitening teeth, usually in gel form, which are typically delivered to the tooth surface by fabricated trays. Such material may be administered under the control of a dental professional or designed for in-home use. These treatments can be considered as the most effective among commercial treatments. The third group includes products designed to be used without the need of a dental professional and can be referred to as “out-of-office” products. Examples include strips, sprays, teeth-wipes, pens, and chewing gum products. A subset of these out-of-office products includes convenience as a main factor distinguishing the products.

In general, the common shortcomings of existing “out-of-office” peroxide-based teeth whitening devices, especially those based on hydrogen or carbamide peroxides, are the inherent instability and lack of teeth-whitening efficacy. The efficacy of known teeth-whitening devices, especially that of chewing gum compositions, deteriorates over time, particularly when exposed to elevated temperatures and humid conditions.

Anhydrous carbamide peroxide is the most effective form of hydrogen peroxide that can be used in chewing gums since it is available in a dry form and is relatively stable in an essentially anhydrous state. However, carbamide peroxide is particularly prone to accelerated decomposition when in contact with water, with other ingredients common to chewing gums such as metal ions from the various particulate matter present in the gum compositions, and with alkaline agents (e.g., calcium carbonate) or other alkaline materials. Goulet, U.S. Pat. No. 5,500,207, teaches a chewing gum comprising carbamide peroxide as the active teeth-whitening agent and is a prime example of one such composition not suitable for commercialization due to the inherent short shelf-life of these un-stabilized carbamide peroxide, and non-achievement of desired teeth-whitening effects. Carbamide peroxide is prone to hydration and once carbamide peroxide becomes hydrated, even to a small degree, it begins to decompose. Since hydration happens readily without proper formulation, packaging and processing, the gum compositions comprising carbamide peroxide do not have the shelf-life or efficacy required for successful commercial products.

With regard to additives that stabilize carbamide peroxides, the current understanding is that the use of stabilizing additives is counterproductive because incorporation of such materials negatively affects the product's whitening efficacy. See, for example, Howard, U.S. Pat. No. 5,824,291, which discloses the use of divalent metal peroxides and an alkaline gum base. These compositions have not proven effective for commercialization, most likely due to the lack of whitening activity, and also due to short shelf life, which is most likely a direct consequence of the absence of a barrier between the alkaline substance (activator) and the peroxide moiety (divalent metal peroxide). In stark contrast to Howard, Orlaowski, et. al. U.S. Pat. No. 6,696,043, teaches a divalent metal peroxide that is activated by fruit acid, but also where there is a barrier between the fruit acid and metal peroxide to keep the metal peroxide from having contact with the fruit acid activator. For activity, the fruit acid and metal peroxide must first make contact, then react together in an aqueous environment (saliva). Here, the peroxide is stabilized by a non-reactive metal carbonate, and even upon “activation” by the fruit acid, does not provide for an effective dosage of “active peroxide” to realize any appreciable teeth whitening.

Microencapsulation has also been employed to aid in the stabilization of carbamide peroxide in chewing gum compositions. Microencapsulation is typically used as a means to protect actives in vitamin and food compositions. In microencapsulation, a barrier material is used to keep water and other materials from contacting the active ingredient and causing unwanted decomposition reactions. For teeth-whiteners, the active agent is typically carbamide peroxide. Gebreselassie (WO/2012/145611 and WO 2006/086061) each teach improved stability of carbamide peroxide in chewing gum compositions by use of microencapsulation technologies. However, these technologies have not yet resulted in an effective teeth-whitening chewing gum.

The requirement of anhydrous conditions places an additional limitation on prior chewing gum compositions known in the art. Since prior art peroxide containing teeth-whitening chewing gum compositions require rigorously anhydrous compositions, they cannot be “extruded,” and require compression using a conventional tablet press to produce the chewing gum composition. These tableted chewing gum compositions suffer from poor mouth feel, since they typically do not have the pleasant characteristics of the well-accepted commercial chewing gum products, which are made by extrusion. Essentially, all commercial chewing gum products in today's marketplace, having blended composition and different shapes, including sticks, coated compositions, etc., are extruded. Extruded gums are more desirable, as they have a much better “mouth-feel” and, thus, are the most popular chewing gums in the marketplace. It is the water content of the “extrudable” chewing gum compositions that gives rise to peroxide containing chewing gum's unsuitability for extrusion. This instability is most likely due to the increased amount of glycerin (or “polyol lubricant”) present in the extruded gum formulations. Glycerin (and propylene glycol) is extremely hygroscopic and always contain some water. As a result, such “extrudable” teeth-whitening chewing gums comprising a peroxide source (carbamide peroxide, metal peroxide, etc.) are not stable, and therefore not commercially viable. This instability is due to decomposition of the carbamide peroxide upon contact with water internal to the chewing gum composition. As a result, extrudable chewing gum compositions for teeth-whitening comprising a peroxide source, and specifically carbamide peroxide, have not been realized commercially.

It is important to also note that teeth-whitening chewing gum compositions known in the art have not been shown to have any appreciable teeth-whitening efficacy. At best, they may be used for maintenance of tooth whiteness or for breath-freshening, not as a primary teeth-whitening device. Additionally, tableted compositions known in the art are problematic for the user due to poor taste and poor mouthfeel and gum activity, especially in direct comparison to their extruded gum counterparts.

Furthermore, abrasives and surfactants, claimed to have teeth-whitening activity, have been described as a substitute for peroxide in chewing gums. For example, Lawlor, WO 02/092027 and Luo, U.S. Pat. No. 6,696,044, each teach a teeth-whitening gum that uses a surfactant as the active whitening agent. Robinson, U.S. Pat. No. 6,416,744, teaches a chewing gum with silica as an abrasive and the active agent for teeth-whitening. However, none of Lawlor, Luo and Robinson provides a composition with any noticeable teeth-whitening activity. Sodium bicarbonate also has been used as a whitening agent. For example, Raksha K. Ballal et al. (Int. J. Clin. Pediatr. Dent. 2016; 9(1):35-38) teaches a gum made with sodium bicarbonate to control pH of the saliva. Teeth-whitening activity of sodium bicarbonate chewing gums is nominal at best since they do not function to chemically remove stains. There are no reports of any significant teeth whitening achieved using a chewing gum comprising chiefly sodium bicarbonate as the active teeth-whitening agent.

Of importance chemotherapeutically, Dr. Keyes (National Institutes of Health) discovered that the use of hydrogen peroxide and baking soda (sodium bicarbonate) is useful as preventative and remedial treatment for periodontal diseases. Others using his technique also noted a teeth-whitening effect. In fact, the whitening effects of hydrogen peroxide aqueous solutions were first reported by the periodontal community in the 1970s. The use of hydrogen peroxide and baking soda as an efficacious combination for teeth-whitening, however, has never been reported, even though the use has been theorized, taught, and practiced as remedy and for the prevention of gingival diseases. Further, based on the available knowledge, sodium bicarbonate is not regarded to be as good an activator as sodium carbonate or potassium carbonate, which are stronger alkaline buffering agents.

One major drawback of the use of oral compositions having hydrogen peroxide in combination with alkaline activating agents, and specifically with carbonate and bicarbonate salts such as baking soda (sodium bicarbonate), is the inherent incompatibility of these agents with hydrogen peroxide. Hydrogen peroxide decomposes quickly in the presence of bases. Sodium bicarbonate is such a base. Known compositions which utilize a hydrogen peroxide source and alkaline activator do not require a barrier between the activator (e.g., bicarbonate ion) and peroxide and thus, the combination is unstable. Also, the strengths of the two actives in gum compositions available in the art are too low for achieving any appreciable teeth whitening in one treatment, and most likely even in multiple treatments.

For a commercial product, it is preferable that the two actives (hydrogen peroxide and the activator) be separated. In other words, a suitable commercial product comprising hydrogen peroxide and alkaline activator preferably has at least two independent parts, separated by a barrier. To this end, Ryles, et. al, U.S. Pat. No. 5,902,568 teaches a teeth-whitening toothpaste-a bi-phasic foam-in which hydrogen peroxide and sodium bicarbonate are stored separately and applied on the toothbrush before use. However, although stable, this toothpaste has not been shown to have any appreciable teeth-whitening effect. The lack of teeth whitening efficacy is due to the low strengths of the two actives and also due to the method of their application to the teeth. Brushing of the teeth with such two-part (or biphasic) composition allows for only a short exposure of the tooth surface to the teeth-bleaching composition, which is inadequate to affect significant teeth whitening.

Thus, even though sodium bicarbonate and hydrogen peroxide have been taught as teeth-whitener, both independently and as a combination, there has been no demonstration of significant improvement in teeth-whitening using products based on these teachings compared to products based on using hydrogen peroxide alone as the bleaching agent. That is, there is no evidence to date that sodium bicarbonate (or any other alkaline activating agent) increases the whitening efficacy of hydrogen peroxide teeth-whiteners. This is possibly due to one or more of: (1) concentrations of hydrogen peroxide and the alkaline activator, e.g., sodium bicarbonate or another alkaline activator used are too low; (2) the device used for applying the combination is not effective; (3) the incipient oral chemistry does not allow full expression of the chemical potential of the combination, and; (4) the time of exposure of the surfaces to the actives is insufficient. These and other shortcomings are the basis for the lack of efficacy of prior art teeth-whitening methods using hydrogen peroxide and an alkaline activator, including those using sodium bicarbonate as the activator.

### BRIEF SUMMARY

As noted above, current teeth-whitening chewing gum formulations suffer from various shortcomings, which may be summarized as follows: (A) the difficulty in formulating chewing gums having appreciable whitening activity and adequate shelf life due to the vulnerability of anhydrous peroxides, particularly carbamide peroxide, in the presence of common ingredients of chewing gum formulations; (B) the difficulty of making peroxide containing chewing gums that allows for extrusion during manufacturing and that also produces gums of adequate shelf-life; (C) technical difficulties associated with manufacturing teeth whitening chewing gums in two parts that mix in the mouth during mastication; (D) the poor whitening effect of teeth-whitening chewing gums of the prior art, which renders them not applicable for primary teeth-whitening, but only as potentially useful for maintenance of teeth previously whitened by traditional teeth-whiteners; (E) unacceptable taste and chewing sensation (“mouth-feel”) of tableted versions of the chewable compositions; and (F) appreciable tooth and gum sensitivity resulting from the use of tableted carbamide peroxide-based tableted teeth whiteners according to prior formulations.

As such, there is a need for a teeth-whitening chewing gum compositions that provide: (1) appreciable whitening efficacy without causing uncomfortable sensitivity to the user; (2) stability (e.g., long shelf-life) in commercially packaged form; (3) consumer acceptable “mouth feel”; and (4) significant bioactivity towards enhancing the microbiological health of the human oral cavity (e.g., a chewing gum that has enough oxidizing power to significantly whiten teeth in one treatment, that also has a shelf life suitable for commercialization). Moreover, there is a need for a bona-fide teeth-whitener that does not cause sensitivity of the soft and hard tissues of the oral cavity unlike other teeth-whiteners comprising carbamide peroxide.

Stable chewable gum-based compositions comprising a peroxide are provided, as well as methods of using the same. A chewing gum that serves to whiten the teeth is the most convenient form of a teeth-whitener since it is easy to carry and travel with, discrete when it is used, and non-intrusive for typical social activities. Chewing gum whitening products offer an attractive alternative to more cumbersome and time intensive whitening systems because they are substantially more user friendly. People find chewing gum pleasurable and are willing to chew gum for much longer periods of time than they typically wear trays or brush their teeth. Such a method of delivery offers whitening action without sacrifice of time or comfort.

It is contemplated that the compositions disclosed herein may include any of the components, or combinations of components, disclosed in U.S. Pat. No. 11,110,045 (“the '045 Patent”), the entire contents of which is incorporated herein in its entirety. For example, the present compositions may include one or more components used in chewable compositions described in the '045 patent, in any in any amount, range, or ratio, disclosed therein. Similarly, the compositions described herein may be used in any of the methods disclosed in the '045 Patent, with or without including any additional components described therein.

In some aspects, stable chewable gum-based compositions comprising a tablet form of carbamide peroxide are provided that (1) have noticeable teeth-whitening properties; (2) a chewing sensation (“mouth-feel”) pleasurable to the human masses; (3) are devoid of teeth and gum sensitivity, and; (4) and renders a healthy condition of the oral microbiology of the user.

Various embodiments of the compositions described herein have a shelf-life suitable for commercialization in the form of packaged goods. Stability is achieved by compounding exclusively with anhydrous ingredients, compounding under dry conditions, compressing the tablet to the proper hardness level, and packaging the tablet in an inert atmosphere and encased in a gas and water impermeable membrane. Also provided are chewable “kit-based” compositions that overcome the taste and mouthfeel shortcomings of known tableted carbamide peroxide gum compositions by including a second gum composition with better taste and mouth-feel properties. In some aspects, compositions according to the disclosure are provided as two pieces of gum, wherein one piece of gum comprises the peroxide chewing gum composition (a “whitener”) and the other comprises a gum base and a source of bicarbonate ion (an “activator”). In some aspects, superior best-mouth feel and taste may be achieved by using an “activator” gum produced using an extrusion process, having a minty sweet flavor. The two gum pieces may be provided as two independent chewable compositions. The composition may in turn be activated by chewing the two gums at the same time (together). In some aspects, the compositions described herein further include one or more gum additives. In some aspect, the peroxide-containing composition is free of metal ions.

In some aspects, described herein is a chewable composition comprising an anhydrous chewing gum base having water content of about 1% by weight or less, a peroxide moiety having water content of about 2% by weight or less, and gum additives.

In some aspects, the, the chewable composition further comprises an abrasive material.

In some aspects, the, “two-piece” chewable compositions are provided which combine any of the tableted peroxide-containing chewable compositions described herein with another chewable composition that comprises a source of bicarbonate ion. In some embodiments, the source of bicarbonate ion is sodium bicarbonate.

Further, a teeth-whitening kit comprising a peroxide containing component (component A) separated from a component comprising a source of bicarbonate ion (component B) is also provided, where the peroxide gum is a tableted gum, and the bicarbonate gum is an extruded gum.

In addition, methods of whitening teeth using the “two-piece” chewable compositions described herein are provided. Such two-gum “kit” chewable compositions are particularly useful in prevention and remediation of many symptoms of oral diseases such as periodontal disease, gingivitis. and halitosis, and maintenance of healthy gingiva and breath. As such, methods for prevention and remediation of the symptoms of these diseases are also provided. Such methods may comprise, e.g., administering an effective amount of any such chewable compositions to a subject in need of treatment for an oral disease, thereby relieving, reducing, or eliminating one or more symptoms of the oral disease. Similarly, methods of maintaining healthy gingiva and breath are contemplated, comprising administering an effective amount of any of the chewable compositions described herein to a subject (e.g., daily, twice-daily, once every other day, or 1, 2, 3, 4, or 4 times per week, to a subject, thereby preventing or reducing the likelihood of the subject developing an oral disease (e.g., halitosis), or otherwise improving or maintaining healthy gingiva.

Aspects of the disclosure will become readily apparent to those skilled in the art from the following detailed description, wherein only exemplary configurations of the teeth-whitening chewable compositions, kits comprising the compositions, and methods for teeth-whitening and prevention and remediation of symptoms of oral diseases using the compositions are described.

As will be realized, the disclosure includes other and different aspects of the disclosed teeth-whitening chewable compositions, kits comprising the compositions, and methods for teeth-whitening and prevention and remediation of symptoms of oral diseases using the compositions. Further, it will be realized that the various details presented throughout this disclosure are capable of modification in various other respects, all without departing from the spirit and scope of the invention. Accordingly, the detailed description and the Examples that follow are to be regarded as illustrative in nature and not as restrictive.

### DETAILED DESCRIPTION

In some aspects, chewable “kit” compositions comprising a two-gum treatment including a tableted peroxide gum (e.g., a “whitener”) and a sodium bicarbonate gum (e.g., an “activator”) at levels effective for teeth-whitening, and oral hygiene, and having shelf life suitable for a commercial packaged goods are described. All ingredients may be essentially anhydrous. The amount of water (by weight) that may be present in the chewable compositions comprising peroxide is typically about 5% or less, preferably about 4% or less and more preferably about 2% or less. Teeth-whitening efficacy is a function of the concentration of the peroxide, the efficacy of activation and duration of exposure of the orally active oxidizing agent to the surfaces of the hard tissue. The amount of peroxide per dosage of the teeth whitening treatment of the present invention is typically about 10-500 mg, (e.g., about 20-400 mg, about 35-350 mg, or about 100-300 mg). The amount of sodium bicarbonate in the present invention is about 20-1,000 mg (e.g., about 40-800 mg, or about 70-700 mg).

The peroxide moiety may be selected from the following: hydrogen peroxide; peroxides generally of the first or second group of the periodic table including calcium peroxide, calcium carbonate peroxide, sodium carbonate peroxide, zinc peroxide, strontium peroxide, other oxygen radical generating agents; and carbamide peroxide.

In the chewable compositions of the present disclosure, the bicarbonate gum (e.g., activator gum) may comprise a pharmaceutically acceptable form of sodium bicarbonate. In some aspects, the bicarbonate gum (e.g., activator gum) is extrudable during manufacture. Such compositions may comprise a wetting agent, e.g., glycerin. Other wetting agents that may be used include ethylene glycol, polyethylene glycol, propylene glycol, polypropylene glycol, fatty acid esters; sorbitol, and combinations or mixtures thereof. The wetting agent may be present, e.g., at about 0.5-5% based on the weight of the chewable composition.

A chewing gum base (“gum base”) may comprise various compositions as shall be described in greater detail below. The amount of chewing gum base in the chewable compositions described herein is typically about 10-50% relative to the weight of the composition.

The compositions according to the disclosure may include one or more gum additives in an amount sufficient to make up 100% of a given composition. The additives may be, e.g., confectionary components such as flavoring agents and surfactants. Xylitol is a flavoring agent of choice and may be used in combination with sucralose or other non-saccharide or non-polyol sweeteners such as aspartame or saccharine to achieve the desired sweetening and flavoring effect. The amount of xylitol, or other polyols such as sorbitol, mannitol, maltitol, etc., that may be used is about 10-70%, e.g., about 15-55%, or about 23-45% relative to the weight of the chewable composition. The amount of sucralose, or other artificial or natural intense sweetening agents such as aspartame, or saccharine (or others including Stevia extracts and other naturally occurring intense sweeteners) that may be used is typically about 0.05-2%, e.g., about 0.1%-1.5%, or about 0.3-1.2% relative to the weight of the chewable composition. Gum additives shall be described in greater detail below.

In some aspects, the composition is free of metal ions.

In some aspects, the present disclosure provides a kit comprised of two different chewable compositions, wherein one piece (the “peroxide” gum piece [‘whitener”]), comprises significant amounts of carbamide peroxide, and the other phase (“activator” gum piece) comprises a composition having sodium bicarbonate as the activator, and a gum base. In some aspects, the activator is a metal bicarbonate salt or a combination of metal bicarbonate salts. In some aspects, the activator is a combination of metal carbonates and bicarbonates. Small amounts of metal carbonate salts (e.g., sodium or potassium carbonate) may be employed to help stabilize the bicarbonate gum composition. The amount of metal carbonate salt that may be used is typically about 0.001-1% (e.g., about 0.01-0.1%, or about 0.02-0.05%) of the weight of the activator phase.

The amount of sodium bicarbonate activator that may be used is typically about 5-65% (e.g., about 5-30%) based on the mass of the second chewable composition (“activator gum”).

In some aspects, the activator is a bicarbonate ion, and a preferred source of bicarbonate ion is sodium bicarbonate. Potassium bicarbonate, a mixture of potassium and sodium bicarbonates, as well as other metal bicarbonates, may also be used alone or in combination in some aspects.

The two gum pieces are typically kept physically separated until they are chewed together. Chewing takes place for a period of time sufficient for completion of the reaction between the active agents of each phase. The preferred time for mastication by the human oral cavity per dosage is from about 1 minute to 60 minutes, more preferably about 5 minutes to about 45 minutes, and most preferably about 30 minutes. In some aspects, the time for mastication may comprise a time within a range of minutes defined by any pair of integers selected from 1-60.

A surprisingly high degree of teeth whitening was observed using the chewable teeth-whitening “kit” compositions described herein. Typically, in the art of teeth-whitening using peroxide, an activator is expected to increase whitening efficacy by about 0%-25% relative to un-activated peroxide. In stark contrast, using the chewable gum compositions described herein, unexpectedly high teeth-whitening, on the order of 100-1000%, was observed.

It was also surprisingly observed that the use of a source of bicarbonate ions (e.g., sodium bicarbonate), as the activator for teeth-whitening led to a significant reduction in sensitivity caused by peroxide. Thus, in some aspects, sodium bicarbonate may be present between 300-400 mg and carbamide peroxide may be present at between 200-300 mg.

In some aspects, the amount of activator in an acceptable formulation according to the present disclosure is an amount that is enough to maintain the pH of the saliva at near neutral or slightly basic during and immediately after chewing both gum compositions simultaneously.

Further, the present disclosure provides for the “whitener” gum piece as a tablet, which can be produced routinely from a tablet press, common to the art of pharmaceutical manufacturing of tablets. Such a tablet comprises essentially the same ingredients in the same relative and absolute amounts as the chewable compositions described herein, optionally with additional ingredients added to aid in the tableting process. In some aspects, a wetting agent will be excluded. Exemplary ingredients include binders, lubricators, coating, etc. Since these ingredients are well known to the pharmaceutical manufacturing art, they are not described here. In a single tablet containing both active agents, peroxide and activator, the agents may be provided in separate layers. The layers (phases) may be separated from each other by a barrier layer, which may be any inert substance that provides physical separation of the two layers. The barrier layer, for example, may be an anhydrous chewable gum composition. Alternatively, it may be a wax. Materials suitable as barrier layers are described in greater detail below.

In some aspects, the gum base composition for the tableted gums is a polymeric material derived from acetate and commercially available. Polymers such as butadiene-styrene, polyvinyl acetate, polyethylene, paraffin, and petroleum waxes are the most prevalent gum bases on the market today.

The acceptable hardness levels for the tablet gums, and specifically to the peroxide containing gum (“whitener”) is typically about 6-20, (e.g., about 8-16 or about 10-14).

In some aspects, the disclosure provides multiphase compositions, wherein the peroxide phase composition may be microencapsulated with an inert micro-membrane. Such membranes are commonly used in the art of food chemistry, for example, for providing vitamin and other active agents in food products to achieve desired shelf-life. See, e.g., Gebreselassie, WO/2012/145611, hereby incorporated by reference, which discloses microencapsulated chewing gum compositions, with details described therein of acceptable microencapsulation technologies.

Teeth-Whitening Kits

In some aspects, the present disclosure provides a teeth-whitening kit comprising a component A (“whitener”) separated from component B (“activator”). Component A may comprise a chewable composition as described herein comprising (1) a peroxide, a gum base, and optionally one or more gum additives; and/or (2) an abrasive material, a wetting agent, a peroxide, a gum base, and optionally one or more gum additives. Component B may comprise a chewable composition comprising a gum base, an activator (or source thereof), and optionally one or more gum additives. The one or more gum additives may be present in any composition described herein in amount necessary to reach 100% of the weight of the composition.

In some kits according to the disclosure, the ratio of the activator (or source thereof):gum base, in percent weight relative to component B, may e.g., be about 5-30:15-50. In some aspects, such kits may further include instructions for use to achieve teeth-whitening.

In some aspects, the activator is bicarbonate ions, and the source of bicarbonate ions is sodium bicarbonate.

In some aspects, component B further comprises a metal carbonate at about 0.001-1% of the weight of component B.

In some aspects, the relative amounts of component A and component B per dose of teeth-whitening treatment is such that the relative amounts of component A and component B per dose is such that pH of a water extract of the composition is at least about 7.

Teeth-Whitening Methods

In some aspects, the present disclosure provides methods for whitening teeth in a human in need thereof. Such methods may comprise (a) placing in the oral cavity of the human a single biphasic composition, or at least two separate compositions (e.g., two chewing gum compositions, one containing a whitener and the other containing an activator); and (b) chewing the composition(s) for about 1 to about 30 minutes. The total weight of the composition may be about 1 g to about 10 g and the chewing may provide about 20-400 mg hydrogen peroxide equivalents (e.g., about 20, 40, 60, 80, 100, 120, 140, 160 180, 200, 220, 240, 260, 280, 300, 320, 340, 360, 380 or 400 mg hydrogen peroxide equivalents, or an amount within a range defined by any pair of the foregoing amounts). In some aspects, such methods may comprise repeating steps (a) and/or (b) multiple times, e.g., 1, 2, 3, 4, or 5 times per day, per week, or per month (or a number of times within a range selected from any pair of the foregoing numbers).

In some aspects, the activator is bicarbonate ions and the source of bicarbonate ion is sodium bicarbonate. In some aspects, the peroxide is carbamide peroxide.

Treatment and/or Remediation of Symptoms of Periodontal Disease

In one aspect, the present disclosure provides methods for treatment or remediation of periodontal disease in a human in need thereof, such methods comprising: (a) placing in the oral cavity of the human a whitener gum piece together with an activator gum piece (or a biphasic composition comprising both components) and (b) chewing the composition(s) for about 1 to about 30 minutes; wherein the total weight of the composition is about 1-10 g and the chewing provides about 20 mg to about 400 mg hydrogen peroxide equivalents (e.g., about 20, 40, 60, 80, 100, 120, 140, 160 180, 200, 220, 240, 260, 280, 300, 320, 340, 360, 380 or 400 mg hydrogen peroxide equivalents, or an amount within a range defined by any pair of the foregoing amounts).

In some aspects, steps (a) and (b) are repeated. For example, the steps are performed one to five times a day for a period of 2-10 days. In some embodiments, the activator is bicarbonate ions and the source of bicarbonate ion is sodium bicarbonate. In some aspects the peroxide is carbamide peroxide. In some embodiments, in the chewable composition, the activator (or source thereof) is present in excess of the carbamide peroxide, and preferably about twice the level, or more the amount of the sodium bicarbonate. In some embodiments, the pH of the saliva of the human during chewing is about 5.5 to about 7.5.

Gum Base

Typically, the gum base contains elastomers (rubbers). The elastomers employed in the gum base will vary greatly depending upon various factors such as the type of gum base desired, the consistency of gum composition desired and the other components used in the composition to make the final chewing gum product. The elastomer may be any water-insoluble polymer known in the art and includes those gum polymers utilized for chewing gums and bubble gums. Illustrative examples of suitable polymers in gum bases include both natural and synthetic elastomers. For example, polymers which are suitable in gum base compositions include, without limitation, natural substances (of vegetable origin) such as chicle, natural rubber, crown gum, nispero, rosidinha, jelutong, perillo, niger gutta, tunu, balata, guttapercha, lechi capsi, sorva, gutta kay, and the like, and combinations thereof. Examples of synthetic elastomers include, without limitation, styrene-butadiene copolymers (SBR), polyisbbutylene, isobutylene-isoprene copolymers, polyethylene, polyvinyl acetate and the like, and combinations thereof. A preferred gum base for use in the chewable compositions described herein is known by the tradename “NOVA-T” and is made by CAFOSA corporation. Another preferred gum base is “PIB-T” made by Eurobase. In some aspects, the gum base is a product sold by CAFOSA under the trade name HiG® 04, HiG® 02, or HiG® 03, or any other gum base known in the art.

Additional useful polymers include: crosslinked polyvinyl pyrrolidone, polymethylmethacrylate; copolymers of lactic acid, polyhydroxyalkanoates, plasticized ethylcellulose, polyvinyl acetatephthalate, and combinations thereof.

The amount of elastomer employed in the gum base may vary depending upon various factors such as the type of gum base used, the consistency of the gum composition desired and the other components used in the composition to make the final chewing gum product.

In some aspects, the gum base may include wax. Wax softens the polymeric elastomer mixture and improves the elasticity of the gum base. When present, the wax(es) employed may have a melting point below about 60° C. (e.g., between about 45° C. and about 55° C.). The low melting wax may be a paraffin wax. The wax may be present in the gum base in an amount from about 6% to about 10% (e.g., about 7% to about 9.5%), by weight of the gum base.

In addition to the low melting point waxes, waxes having a higher melting point may be used in the gum base in amounts up to about 5%, by weight of the gum base. Such high melting waxes include beeswax, vegetable wax, candelilla wax, carnuba wax, most petroleum waxes, and the like, and mixtures thereof.

In addition to the components set out above, the gum base may include a variety of other ingredients, such as components selected from elastomer solvents, emulsifiers, plasticizers, fillers, and mixtures thereof.

The gum base may contain elastomer solvents to aid in softening the elastomer component. Such elastomer solvents may include those elastomer solvents known in the art, for example, terpinene resins such as polymers of alpha-pinene or beta-pinene, methyl, glycerol and pentaerythritol esters of rosins and modified rosins and gums such as hydrogenated, dimerized and polymerized rosins, and mixtures thereof. Examples of elastomer solvents suitable for use herein include the pentaerythritol ester of partially hydrogenated wood and gum rosin, the pentaerythritol ester of wood and gum rosin, the glycerol ester of wood rosin, the glycerol ester of partially dimerized wood and gum rosin, the glycerol ester of polymerized wood and gum rosin, the glycerol ester of tall oil rosin, the glycerol ester of wood and gum rosin and the partially hydrogenated wood and gum rosin and the partially hydrogenated methyl ester of wood and rosin, and the like, and mixtures thereof. The elastomer solvent may be employed in the gum base in amounts from about 2% to about 15%, and preferably from about 7% to about 11%, by weight of the gum base.

The gum base may also include emulsifiers which aid in dispersing the immiscible components into a single stable system. The emulsifiers useful in this invention include glyceryl monostearate, lecithin, fatty acid mono-glycerides, diglycerides, propylene glycol monostearate, and the like, and mixtures thereof. The emulsifier may be employed in amounts from about 2% to about 15%, and more specifically, from about 7% to about 11%, by weight of the gum base.

The gum base may also include plasticizers or softeners to provide a variety of desirable textures and consistency properties. Because of the low molecular weight of these ingredients, the plasticizers and softeners can penetrate the fundamental structure of the gum base making it plastic and less viscous. Useful plasticizers and softeners include lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, and mixtures thereof. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, mixtures thereof, and the like, may also be incorporated into the gum base. The plasticizers and softeners are generally employed in the gum base in amounts up to about 20% by weight of the gum base, and more specifically in amounts from about 9% to about 17%, by weight of the gum base.

Plasticizers also include are the hydrogenated vegetable oils and include soybean oil and cottonseed oil which may be employed alone or in combination. These plasticizers may provide the gum base with good texture and soft chew characteristics. These plasticizers and softeners are generally employed in amounts from about 5% to about 14%, and more specifically in amounts from about 5% to about 13.5%, by weight of the gum base.

Anhydrous glycerin may also be employed as a softening agent, such as the commercially available United States Pharmacopeia (USP) grade. Glycerin is a syrupy liquid with a sweet warm taste and has a sweetness of about 60% of that of cane sugar. Because glycerin is hygroscopic, the anhydrous glycerin may be maintained under anhydrous conditions throughout the preparation of the chewing gum composition.

In some embodiments, the gum base of this invention may also include effective amounts of bulking agents such as mineral adjuvants which may serve as fillers and textural agents. Useful mineral adjuvants include calcium carbonate, magnesium carbonate, alumina, aluminum hydroxide, aluminum silicate, talc, tricalcium phosphate, dicalcium phosphate, calcium sulfate and the like, and mixtures thereof. These fillers or adjuvants may be used in the gum base compositions in various amounts. The amount of filler, may be present in an amount from about zero to about 40%, and more specifically from about zero to about 30%, by weight of the gum base. In some embodiments, the amount of filler will be from about zero to about 15%, more specifically from about 3% to about 11%.

A variety of traditional ingredients may be optionally included in the gum base in effective amounts such as coloring agents, antioxidants, preservatives, flavoring agents, and the like. For example, titanium dioxide and other dyes suitable for food, drug and cosmetic applications, known as F. D. & C. dyes, may be utilized. An anti-oxidant such as butylated hydroxytoluene (BHT), butylated hydroxyanisole (BFIA), propyl gallate, and mixtures thereof, may also be included. Other conventional chewing gum additives known to one having ordinary skill in the chewing gum art may also be used in the gum base.

Abrasive Materials

Suitable abrasive materials include, e.g., silicas, aluminas, phosphates, carbonates and combinations thereof. In some embodiments, the abrasive agent is a silica selected from: precipitated amorphous silica, silica gels, and combinations thereof. In some embodiments the abrasive material is selected from the following: calcium carbonate, sodium bicarbonate, sodium metaphosphate, potassium metaphosphate, tricalcium phosphate, dehydrated dicalcium phosphate, and combinations thereof.

The abrasive material contemplated for use in the chewable compositions described herein may be any material which does not excessively abrade dentin. However, silica dental abrasives have unique benefits of exceptional dental cleaning and polishing performance without unduly abrading tooth enamel or dentin. Silica is a preferred abrasive agent for the peroxide containing composition (or phase/layer) of the present compositions/kits. Other suitable abrasive materials, including aluminas, phosphates, carbonates and combinations thereof, may be employed as abrasive agents in the bicarbonate containing composition (or phase/layer) of the present invention.

The silica abrasive materials described herein, as well as other abrasive material, generally have an average particle size ranging between about 0.1 to about 30 microns, and preferably from about 5 to about 15 microns. The abrasive can be precipitated amorphous silica or silica gels such as the silica xerogels described in U.S. Pat. No. 3,538,230 to Pader, et al. and U.S. Pat. No. 3,862,307 to DiGiulio, both incorporated herein by reference in their entireties. Preferred are the silica xerogels marketed under the trade name “Syloid” by the W.R. Grace & Company, Davison Chemical Division. Also preferred are the precipitated amorphous silica materials, such as those marketed by the J. M. Huber Corporation under the trade name “Zeodent”, particularly the silica carrying the designation “Zeodent 113”. The types of silica dental abrasives useful in the present invention are described in detail in U.S. Pat. No. 4,340,583 to Wason, incorporated herein by reference in its entirety.

Gum Additives

Additives conventionally used in chewing gums are known to a person skilled in the art of manufacturing chewing gums and may be used in the compositions described herein. Such additives include bulk sweeteners, high intensity sweeteners, flavoring agents, softeners, emulsifiers, coloring agents, binding agents, acidulants, fillers, antioxidants, and other components such as pharmaceutically or biologically active substances, that confer desired properties to the finished chewing gum product.

Examples of suitable sweeteners are listed in the following. Suitable bulk sweeteners include, e.g., both sugar and non-sugar components. Useful sugar sweeteners are saccharide-containing components commonly known in the chewing gum art including, but not limited to, sucrose, dextrose, maltose, dextrins, trehalose, D-tagatose, dried invert sugar, fructose, levulose, galactose, corn syrup solids, and the like, alone or in combination. Sorbitol can be used as a non-sugar sweetener. Other useful non-sugar sweeteners include, but are not limited to, other sugar alcohols such as mannitol, xylitol, hydrogenated starch hydrolysates, maltitol, isomaltol, erythritol, lactitol and the like, alone or in combination. High intensity artificial sweetening agents can also be used alone or in combination with the above sweeteners. Preferred high intensity sweeteners include, but are not limited to sucralose, aspartame, salts of acesulfame, alitame, saccharin and its salts, neotame, cyclamic acid and its salts, glycyrrhizin, dihydrochalcones, thaumatin, monellin, sterioside and the like, alone or in combination. In order to provide longer lasting sweetness and flavour perception, it may be desirable to encapsulate or otherwise control the release of at least a portion of the artificial sweetener. Techniques such as wet granulation, wax granulation, spray drying, spray chilling, fluid bed coating, coascervation, encapsulation in yeast cells and fibre extrusion may be used to achieve desired release characteristics. Encapsulation of sweetening agents can also be provided e.g. using as the encapsulation agent another chewing gum component such as a resinous compound.

If a low-calorie gum is desired, a low-caloric bulking agent can be used. Examples of low caloric bulking agents include polydextrose, Raftilose, Raffilin, Inuline, fructooligosaccharides (NutraFlora®), palatinose oligosaccharide; guar gum hydrolysates (e.g., Sun Fiber®) or indigestible dextrins (e.g. Fibersol®). However, other low-calorie-bulking agents can also be used.

Further chewing gum additives which may be included in the chewing gum mixture processed in the present process include surfactants and/or solubilizers. Anionic, cationic, amphoteric or non-ionic solubilizers can be used. Suitable solubilizers include lecithins, polyxyethylene stearate, polyoxyethylene sorbitan fatty acid esters, fatty acid salts, mono and diacetyl tartaric acid esters of mono and diglycerides of edible fatty acids, citric acid esters of mono and diglycerides of edible fatty acids, saccharose esters of fatty acids, polyglycerol esters of fatty acids, polyglycerol esters of interesterified castor oil acid (E476), sodium stearoyllatylate, sodium lauryl sulfate and sorbitan esters of fatty acids and polyoxyethylated hydrogenated castor oil (e.g., the product sold under the trade name CREMOPHOR®), block copolymers of ethylene oxide and propylene oxide (e.g. products sold under trade names PLURONIC® and POLOXAMER®), polyoxyethylene fatty alcohol ethers, polyoxyethylene sorbitan fatty acid esters, sorbitan esters of fatty acids and polyoxyethylene steraric acid esters.

Particularly suitable solubilizers include polyoxyethylene stearates, such as for instance polyoxyethylene(S) stearate and polyoxyethylene(40)stearate, the polyoxyethylene sorbitan fatty acid esters sold under the trade name TWEEN®, for instance TWEEN 20 (monolaurate), TWEEN 80 (monooleate), TWEEN 40 (monopalmitate), TWEEN 60 (monostearate) or TWEEN 65 (tristearate), mono and diacetyl tartaric acid esters of mono and diglycerides of edible fatty acids, citric acid esters of mono and diglycerides of edible fatty acids, sodium stearoyllactylate, sodium laurylsulfate, polyoxyethylated hydrogenated castor oil, block-copolymers of ethylene oxide and propyleneoxide and polyoxyethylene fatty alcohol ether. The solubilizer may either be a single compound or a combination of several compounds.

Aroma agents and flavoring agents which are useful in the chewable compositions described herein include natural and synthetic flavorings in the form of freeze-dried natural vegetable components, essential oils, essences, extracts, powders, including acids and other substances capable of affecting the taste profile. Examples of liquid and powdered flavorings include coconut, coffee, chocolate, vanilla, grapefruit, orange, lime, menthol, licorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

Various synthetic flavors, such as mixed fruit flavor may also be used. Sorbitol can be used as a non-sugar sweetener. Other useful non-sugar sweeteners include, but are not limited to, other sugar alcohols such as mannitol, xylitol, hydrogenated starch hydrolysates, maltitol, iso-maltol, erythritol, lactitol and the like, alone or in combination.

### EXAMPLES

### Example 1: A Teeth Whitener Kit

This example illustrates an exemplary carbamide peroxide gum (“whitener”) prepared by tablet press technology, and sodium bicarbonate gum (“activator”) prepared by extrusion technology.

Whitener Gum Component

TABLE 1
Composition of the whitener
gum component used in this example.

| Ingredient | Amount |
|---|---|
| Carbamide peroxide Gum base | 250 mg |
| Xylitol Sorbitol | 900 mg |
| Peppermint oil (spray-dried) Sucralose | 850 mg |
| Magnesium Stearate Stearic Acid | 500 mg |
| Microcrystalline Cellulose | 25 mg |
| Carbamide peroxide Gum base | 30 mg |
| Xylitol Sorbitol | 75 mg |
| Peppermint oil (spray-dried) Sucralose | 100 mg |
| Magnesium Stearate Stearic Acid | 125 mg |
| Microcrystalline Cellulose | 250 mg |

The above-listed ingredients were combined and blended in a ribbon blender at slow speed until homogeneous, then submitted immediately to a tablet press. Circular tablets were ejected at about 2.6 grams per composition.

Activator Gum Component

TABLE 2
Composition of the activator gum
component used in this example.

| Ingredient | Percent |
|---|---|
| Base PIB | 27.00 |
| Base Nova-T | 5.00 |
| Lecithin, liquid | 0.20 |
| Titanium Dioxide | 0.50 |
| Multisweet 3145 Lycasin | 8.00 |
| Xylitol CM90 | 35.60 |
| Sodium Bicarbonate | 15.00 |
| Silicon Dioxide | 5.00 |
| Glycerine | 0.40 |
| Sucralose | 0.50 |
| WS-23 Coolant | 0.30 |
| Nat. Peppermint Oil | 1.50 |
| flavorchem | |
| Cool Ice Flavor US-6984 | 0.60 |
| Menthol Crystals | 0.40 |

Manufacturing Procedure
1) Add gum bases, ½ of the Maltitol Syrup, 1 tray of Xylitol and Titanium Dioxide and mixed scrap (if available) to warm mixer and start blades.
2) When base is soft slowly add ½ of remaining Xylitol and mix for approx. 2 minutes. 3) Add coolant, Sucralose and Sodium Bicarbonate/Silicon Dioxide mixture and mix approx 2 minutes.
4) Add Glycerin and remaining ½ Maltitol Syrup and mix approx 1.5 minutes.
5) Slowly add remaining Xylitol and mix approx 2 minutes.
6) Add liquid flavors and mix 2-3 minutes until batch is uniform. Record final batch temperature.

Use/Application

Two pieces of gum, one Whitener gum piece and one Activator gum piece, are chewed together for 5-30 minutes, then expulsed. Noticeable teeth whitening was recorded, and much improved smelling breath and overall oral cleaning effect were also immediately apparent.

### Example 2: Teeth Whitener Kit—Chewable Gum

Whitener Gum Component

TABLE 3
Composition of the chewable gum
whitener component used in this example.

| Ingredient | Amount (g) |
|---|---|
| Carbamide peroxide | 100 |
| Gum base (HIG PWD-04-L020323214) | 650 |
| Xylitol | 100 |
| Sorbitol | 100 |
| Peppermint oil (spray-dried) Sucralose | 0.5 |
| Magnesium Stearate | 15 |
| Stearic Acid | 5 |
| Microcrystalline Cellulose | 5 |

The above-listed ingredients were combined and blended in a ribbon blender at slow speed until homogeneous, then submitted immediately to a tablet press. Circular tablets were ejected at about 2.6 grams per composition.

Whitener Gum Component

TABLE 4
Composition of the chewable gum activator
component used in this example.

| Ingredient | Percent |
|---|---|
| Base PIB | 27.000 |
| Base Nova-T | 5.500 |
| Lecithin, liquid | 0.200 |
| Multisweet 3145 Lycasin | 8.000 |
| Xylitol CM90 | 35.600 |
| Sodium Bicarbonate | 17.000 |
| Silicon Dioxide | 3.000 |
| Glycerine | 0.400 |
| Sucralose | 0.500 |
| WS-23 Coolant | 0.300 |
| Nat. Peppermint Oil | |
| flavorchem | 1.500 |
| Cool Ice Flavor US-6984 | 0.600 |
| Menthol Crystals | 0.400 |

Manufacturing Procedure:
1) Add gum bases, ½ of the Maltitol Syrup, 1 tray of Xylitol and Titanium Dioxide and mixed scrap (if available) to warm mixer and start blades.
2) When base is soft slowly add ½ of remaining Xylitol and mix for approx. 2 minutes. 3) Add coolant, Sucralose and Sodium Bicarbonate/Silicon Dioxide mixture and mix approx 2 minutes.
4) Add Glycerin and remaining ½ Maltitol Syrup and mix approx 1.5 minutes.
5) Slowly add remaining Xylitol and mix approximately 2 minutes.

### Example 3: Whitener, Chewable Gum Composition Made by Tablet Press Technology

Whitener Gum Component

TABLE 5
Composition of the chewable gum whitener
component used in this example.

| Ingredient | Amount (g) |
|---|---|
| Carbamide peroxide | 100 |
| Gum base (HIG PWD-04-L020323214) | 870 |
| Magnesium Stearate | 15 |
| Silica | 15 |

Manufacturing Procedure: The above-listed ingredients were combined and blended in a ribbon blender at slow speed until homogeneous, then submitted immediately to a tablet press. Circular tablets were ejected at about 2.6 grams per composition at a hardness between 8-16.

Activator Gum Component

TABLE 6
Composition of the chewable gum activator
component used in this example.

| Ingredient | Percent |
|---|---|
| Base PIB | 27.000 |
| Base Nova-T | 5.500 |
| Lecithin, liquid | 0.200 |
| Multisweet 3145 Lycasin | 8.000 |
| Xylitol CM90 | 35.600 |
| Sodium Bicarbonate | 17.000 |
| Silicon Dioxide | 3.000 |
| Glycerine | 0.400 |
| Sucralose | 0.500 |
| WS-23 Coolant | 0.300 |
| Nat. Peppermint Oil | 1.500 |
| flavorchem | |
| Cool Ice Flavor US-6984 | 0.600 |
| Menthol Crystals | 0.400 |

Manufacturing Procedure
1) Add gum bases, ½ of the Maltitol Syrup, 1 tray of Xylitol and Titanium Dioxide and mixed scrap (if available) to warm mixer and start blades.
2) When base is soft slowly add ½ of remaining Xylitol and mix for approx. 2 minutes
3) Add coolant, Sucralose and Sodium Bicarbonate/Silicon Dioxide mixture and mix approx 2 minutes.
4) Add Glycerin and remaining ½ Maltitol Syrup and mix approx 1.5 minutes.
5) Slowly add remaining Xylitol and mix approx 2 minutes.
6) Add liquid flavors and mix 2-3 minutes until batch is uniform. Record final batch temperature.

Use/Application: Two pieces of gum, one Whitener gum piece and one Activator gum piece, are chewed together for 5-30 minutes, then expulsed. Noticeable teeth whitening was recorded, and much improved smelling breath and overall oral cleaning effect were also effected and immediate.

1. A teeth-whitening kit, comprising a component A separated from a component B, wherein component A is a first chewable composition, comprising an anhydrous chewing gum base having water content of about 1% or less,
one or more anhydrous tableting excipients,
carbamide peroxide having water content of about 2% or less, and
one or more gum additives;
wherein the ratio of the gum base:the carbamide peroxide: the one or more tableting excipients, in percent weight relative to the first chewable composition, is about 10-50:1-20:0.5-8, and the one or more gum additives are present in an amount sufficient to reach 100% of the weight of the first chewable composition; and
wherein component B is a second chewable composition, comprising a source of a bicarbonate ion, a gum base, and one or more gum additives, wherein the source of the bicarbonate ion and the gum base, respectively, are present in a ratio of about 5-30: 15-50 in percent weight relative to the weight of component B, and the one or more gum additives are present in an amount sufficient to reach 100% of the weight of the second chewable composition.

2. The kit of claim 1, wherein the source of the bicarbonate ion is sodium bicarbonate.

3. The kit of claim 1, wherein component B further comprises a metal carbonate at about 0.001-1% of the weight of component B.

4. The kit of claim 2, wherein the relative amounts of component A and component B per dose of teeth-whitening treatment is such that sodium bicarbonate is present at three times or more compared to the amount of the tablet excipient.

6. The kit of claim 1, wherein the tablet excipient is comprised of magnesium stearate and/or silicon dioxide.

8. A method for whitening teeth of a human subject in need thereof, the method comprising:
placing in the oral cavity of the human subject a single chewable composition comprising a first and a second region separated by a barrier layer,
wherein the first region is a first chewable composition comprising
an anhydrous chewing gum base having water content of about 1% or less,
one or more anhydrous tablet excipients,
carbamide peroxide having water content of about 2% or less, and
one or more gum additives,
wherein, the ratio of the gum base: the carbamide peroxide: the one or more tablet excipients, in percent weight relative to the first chewable composition, is about 10-50: 1-20: 0.5-8, and the one or more gum additives are present in an amount sufficient to reach 100% of the weight of the first chewable composition; and
the second region is a second chewable composition comprising a source of bicarbonate ion, a gum base, and one or more gum additives, wherein the source of bicarbonate ion and the gum base, respectively, are present in a ratio of about 5-30:15-50 in percent weight relative to the second chewable composition, and the one or more gum additives are present in an amount sufficient to reach 100% of the weight of the second chewable composition; and
chewing the single chewable composition,
wherein the total weight of the single chewable composition is about 1 g to about 10 g and the chewing provides about 20 mg to about 400 mg hydrogen peroxide equivalents.

9. The method of claim 8, wherein the source of bicarbonate ion is sodium bicarbonate.

10. The method of claim 9, wherein, in the single chewable composition, the ratio of tablet excipient:carbamide peroxide:sodium bicarbonate, by mass is about 0.5-5: 5-20: 10-35.

11. The method of claim 8, wherein the second chewable composition further comprises a metal carbonate at about 0.001-1% of the weight of the second region.

12. The method of claim 8, wherein the tablet excipient is magnesium stearate and/or silicon dioxide.

13. The method of claim 9, wherein, in the single chewable composition, sodium bicarbonate is present at three times or more the amount of the tablet excipient.

14. The method of claim 8, wherein the pH of the saliva of the human subject during the chewing step is about 5.5 to about 7.

15. The method of claim 8, wherein the single chewable composition is chewed for 1 to 30 minutes during the chewing step.

16. A method for whitening teeth of a human subject in need thereof, the method comprising:
placing in the oral cavity of the human subject a first composition and a second composition, and
chewing together the first and second compositions;
wherein the first composition is a chewable composition comprising an anhydrous chewing gum base having water content of about 1% or less, one or more tablet excipients having water content 1% or less, carbamide peroxide having water content of about 2% or less, and one or more gum additives;
wherein the ratio of the gum base: the carbamide peroxide: the one or more tablet excipients, in percent weight relative to the first composition, is about 10-50: 1-20: 0.5-8, and the one or more gum additives are present in an amount sufficient to reach 100% of the weight of the first composition; and
the second composition is a chewable composition comprising a source of bicarbonate ion, a gum base, and one or more gum additives, wherein the source of bicarbonate ion and the gum base are present, respectively, in a ratio of about 5-30: 15-50 in percent weight relative to the second composition, and the one or more gum additives are present at an amount sufficient to reach 100% of the weight of the second composition, and
wherein the total weight of the first and the second composition is about 1 g to about 10 g and the chewing provides about 20 mg to about 400 mg hydrogen peroxide equivalents.

17. The method of claim 16, wherein the source of bicarbonate ion is sodium bicarbonate.

18. The method of claim 17, wherein in the first and the second chewable composition combined, the ratio of the one or more tablet excipients: the carbamide peroxide: the sodium bicarbonate, by mass is about 0.5-5: 5-20: 10-35.

19. The method of claim 16, wherein the second composition further comprises a metal carbonate at about 0.001-1% of the weight of the second composition.

20. The method of claim 16, wherein the one or more tablet excipients comprises a combination of magnesium stearate and silicon dioxide.

21. The method of claim 17, wherein, sodium bicarbonate is present at three times or more the amount of the tablet excipient.

22. The method of claim 16, wherein pH of the saliva of the human subject during the chewing step is about 5.5 to about 7.

23. The method of claim 16, wherein the chewing step is performed for 1 to 30 minutes.
